SEQUENCE LISTING (49 sequences)

SEQ ID NO: 1 - DNA, 2760 bp, Brassica napus
atgcgagtcgtcgacactcgtcgttaccaaaagggagaaaagtcatacgaagaaaataataataaaccggagaagaagttggaaaaaagaacaaaaacaaaaagacggaaaagaaatttgtggtggggggagactgtatatagtctgataaggcgggcatgcaaattatgttcttcacagtttccattttcatgcgtaaagaatatgaaaggctctgtgaaatcatttagtctcattcctatttccttttgttttctcttcttatttcgtgatgagtttgcggttcctgctaggcacttgtgccatcctcaacaaagggaagcaattctggagttcaaaaacgagttccagattcagaagccttgttccggctggacggtgtcatgggtgaataacagcgactgctgttcttgggatggtatcgcatgtgatgccacttttggggatgtgattgagctgaaccttggtggcaactgcatccatggcgagctcaattccaaaaacactattttgaagcttcaaagtcttccttttttagaaactctcaaccttgcaggcaattatttcagtggtaacattccatcttcgcttggaaatctttctaagctaaccactcttgatctttcagataatgcttttaatggtgaaattccatcttcacttggaaagctttataacctcaccattctcaatctctcccacaacaaacttattggtaaaatcccatcttcttttggcagattgaaacatctcaccggcttatatgctgcagacaacgagcttagtggtaactttcctgttacgacgctactaaatctaacaaaattgttatctttatcactctatgataaccagttcacaggcatgcttccacctaacataagctcactctccaacttggtggccttttacatacgtggcaacgctttaactggaactcttccttcttccctctttagcatcccttctttgctttacgttactttggaaggtaaccaactaaacggtacacttgattttgggaacgtatcttcatcatcaaagctaatgcaactacgccttgggaataacaatttcttgggatcgattcccagagccatctccaaattagtcaaccttgctacacttgacctttcgcatctcaacacccaaggcttagcacttgaccttagcattctctggaatctcaagtcactggaagaactcgacatctccgacttgaacaccaccactgcgattgacttgaatgctatcttatcacgatacaagtggctggataaactgaatctcacgggaaaccacgttacgtatgaaaagcgaagctcagtatcagatcctccgttattaagcgagctgtacttgtcaggatgcagattcaccaccgggtttccggagctcctgcgaacccaacacaacatgaggacactagacatttccaacaacaaaatcaaaggtcaagtccctggatggttatgggagctatcaactctagaatacctgaatatctccaacaacactttcaccagtttcgaaaatccgaaaaaactccggcaaccatcctctctggaatacctttttggcgccaacaacaatttcacgggcaggatccccagtttcatatgtgagttgcgatctctcaccgttctcgatttatccagcaacaaattcaacggttcattaccacgttgtatcggaaagttcagtagtgttctcgaagctttaaatcttcgacaaaaccgtctcagtggacgtcttccaaagattatattcagaagtttaacgtcgtttgacattggtcataacaaactggttggaaagcttccaagatctttgatcgctaactcttctcttgaagttttgaatgtggaaagcaacagattcaacgacacgtttccatcctggctgagttctctccccgagctacaagttcttgtccttcgctccaatgcgttccacggaccagtacaccaaactcggttttctaaactgcggatcattgatatatcgcataatcggttcagcggaatgttgccatcgaacttctttctgaactggacagcaatgcactctattggaaaagatggagatcaatctaacggaaactatatgggtacatactattattttgattcaatggttttgatgaacaaaggtgtagagatggagctggtacgtatcttaacaatctacacagcgctagacttttcggagaacgaattcgaaggagtgattccatcgtccatcggtttgttgaaagagcttcacgtgctcaacttatcaggcaatgcgttcactggccgtatcccgtcgtctatggggaacctctcgtctctggaatcactggacctttccagaaacaagctcacgggagcaattccacaagagctagggaacctctcctaccttgcttacatgaacttctctcataaccagcttgtgggtctagtgccagggggcactcagtttcggacgcagccatgcagttctttcaaggacaacccgggactttttggcccttcgcttgaagaagtttgtgtagatcatatccacgggaaaacatcacaaccgtctgaaatgtcaaaggaagaagaagatggccaagaggaggtgataagttggatagcagctgcaattggtttcatacctggtattgtatttggattcacgatgggatacataatggtttcctacaaaccagagtggtttataaacctttttggccgaactaaacgcagaaggataagcaccacacgtcgttaa

SEQ ID NO: 2 - DNA, 2853 bp, Brassica napus
atgaaaggctctgtgaaatcatttagtctcattcctatttccttttgttttctcttcttatttcgtgatgagtttgcggttcctgctagacacttgtgccatcctcagcaaagggaagcaattctggagttgaagaacgagttccatattcagaagccttgttctgacgaccggacggtgtcatgggtgaataacagcgactgttgttcttgggatggtatcagatgtgatgccacttttggggatgtgattgagctgaaccttggtggcaactgcatccatggcgagctcaattccaaaaacactattttgaagcttcaaagtcttccttttttagcaactctcgacctttcagacaattatttcagtggtaacattccatcttcgcttggaaatctttctaagctaaccactcttgatctttcagataatgattttaatggtgaaatcccatcttcacttggaaacctttctaacctcaccactcttgatctttcatataatgcttttaatggtgaaatcccatcttcacttggaaacctttctaacctcaccattctcaaactctcccagaacaaacttattggtaaaatcccaccttcacttggaaacctttcgtatctcacccatcttacactttgtgctaacaacttggttggtgaaattccatactcacttgcaaacctttctcatcatctcacctttcttaatatatgcgaaaacagtttttctggtgaaattccatcattcttgggaaatttttcacttctgaccttactcgatctctcagcaaacaatttcgtcggggaaatcccatcttctttcggcagattgaaacatctcaccatcttatccgctggagaaaacaagcttactggtaactttcctgttacgctactaaatctaacaaaattgttagatttatcacttggttacaaccagttcacaggcatgcttccacctaacgtaagcttactctccaacttggaggccttttccataggtgggaacgctttaactggaactcttccttcttccctctttagcatcccttctttgacttacgttagtttggaaaataaccaactaaacggtacacttgattttgggaacgtatcttcatcatcaaagctaatgcaactacgccttgggaataacaacttcttgggatcgattcccagagccatctccaaattagtcaaccttgatacacttgacctttcgcatctcaacacccaaggctcatcagttgaccttagcattctctggaatctcaagtcactcgtagaactcgacatctccgacttgaacaccaccactgcgattgacttgaatgatatcttatcacgtttcaagtggctggatacactgaatctcacgggaaaccacgttacgtatgaaaagagaatttcagtttcagatcctccgttattaagagatctgtacttgtcaggatgcagattcaccaccgagtttccagggttcatacgaactcaacacaacatggaagcactagacatttccaacaacaaaatcaaaggtcaagtccctggatggttatgggagctatcaactctatattacctgaatctctccaacaacactttcaccagtttcgaaagtccgaataaactccggcaaccatcctctctgtattacttttccggcgccaacaacaatttcacgggcgggatccccagtttcatatgtgagttgcactctctaatcattctcgatttatctagcaacagattcaacggttcattaccgcgttgtgtcggaaagttcagtagtgtccttgaagctctaaatcttagacaaaatcgtctcagcggacgtcttccaaagaagattatatcaagaggtctaaagtctttggacatcggtcataacaaactggttggaaagcttccgagatctttgatcgctaactcttctcttgaagttttgaatgtggaaagcaacagattcaacgacacgtttccatcctggttgagttctctccccgagctacaagttcttgtccttcgctccaatgcgttccatggaccgatacatcaaactcggttttataaactgcgaatcatagacatatcgcataatcggttcaacggaacgttgccgttggacttctttgtgaactggacatcaatgcactttatcggaaaaaatggagttcaatctaacggaaactacatgggtactagaagatattattttgattcaatggttttgatgaataaaggcatagagatggagctggtacgtatcttatatatctacactgcgctagacttttcggagaacgaattcgaaggagtgattccatcgtccatcggtttgttgaaagagcttcacgtgctcaacttatcaggcaatgcgttcactggccgtatcccgtcgtctatggggaacctctcgtctctcgagtcactggacctttccagaaacaagcttacaggagaaattccgcaagagctagggaacctctcctaccttgcttacatgaacttctctcataaccagcttgtgggtctagtgccagggggcactcagtttcggacgcagccttgcagttctttcaaggacaacccgggactttttggcccttcacttaatcaagcttgtgtagatatccacgggaaaacatcacaaccgtctgaaatgtcaaaggaagaagaagaagatggccaagaggaggtgataagttggatagcagctgcaattggtttcatacctggtattgcatttggattcacgatggaatacataatggtttcctacaaaccagagtggtttataaacctttttggccgaaccaaacgcagaaggataagcaccacacgtcgttaa

SEQ ID NO: 3 - DNA, 2853 bp, Brassica napus; identical to SEQ ID NO: 2

SEQ ID NO: 4 - protein, 918 aa, Brassica napus
MRVVDTRRYQKGEKSYEENNNKPEKKLEKRTKTKRRKRNLWWGETVYSLIRRACKLCSSQFPFSCVKNMKGSVKSFSLIPISFCFLFLFRDEFAVPARHLCHPQQREAILEFKNEFQIQKPCSGWTVSWVNNSDCCSWDGIACDATFGDVIELNLGGNCIHGELNSKNTILKLQSLPFLETLNLAGNYFSGNIPSSLGNLSKLTTLDLSDNAFNGEIPSSLGKLYNLTILNLSHNKLIGKIPSSFGRLKHLTGLYAADNELSGNFPVTTLLNLTKLLSLSLYDNQFTGMLPPNISSLSNLVAFYIRGNALTGTLPSSLFSIPSLLYVTLEGNQLNGTLDFGNVSSSSKLMQLRLGNNNFLGSIPRAISKLVNLATLDLSHLNTQGLALDLSILWNLKSLEELDISDLNTTTAIDLNAILSRYKWLDKLNLTGNHVTYEKRSSVSDPPLLSELYLSGCRFTTGFPELLRTQHNMRTLDISNNKIKGQVPGWLWELSTLEYLNISNNTFTSFENPKKLRQPSSLEYLFGANNNFTGRIPSFICELRSLTVLDLSSNKFNGSLPRCIGKFSSVLEALNLRQNRLSGRLPIIFRSLTSFDIGHNKLVGKLPRSLIANSSLEVLNVESNRFNDTFPSWLSSLPELQVLVLRSNAFHGPVHQTRFSKLRIIDISHNRFSGMLPSNFFLNWTAMHSIGKDGDQSNGNYMGTYYYFDSMVLMNKGVEMELVRILTIYTALDFSENEFEGVIPSSIGLLKELHVLNLSGNAFTGRIPSSMGNLSSLESLDLSRNKLTGAIPQELGNLSYLAYMNFSHNQLVGLVPGGTQFRTQPCSSFKDNPGLFGPSLEEVCVDHIHGKTSQPSEMSKEEEDGQEEVISWIAAAIGFIPGIVFGFTMGYIMVSYKPEWFINLFGRTKRRRISTTRR

SEQ ID NO: 5 - DNA, 20 bp, Artificial Sequence
  Primer
ggtatcgcattctgtgacta

SEQ ID NO: 6 - DNA, 20 bp, Artificial Sequence
  Primer
ggagatgtgcttcaacgtga

SEQ ID NO: 7 - DNA, 18 bp, Artificial Sequence
  Primer
acattcttgggccgtagg

SEQ ID NO: 8 - DNA, 20 bp, Artificial Sequence
  Primer
gacaaacacaatggactcaa

SEQ ID NO: 9 - DNA, 20 bp, Artificial Sequence
  Primer
gaggtagagaaagacgaaga

SEQ ID NO: 10 - DNA, 21 bp, Artificial Sequence
  Primer
atcgtttaaggaatgtgccaa

SEQ ID NO: 11 - DNA, 17 bp, Artificial Sequence
  Primer
ccacagtttctggagac

SEQ ID NO: 12 - DNA, 20 bp, Artificial Sequence
  Primer
gtagcaaaggaatcaattaa

SEQ ID NO: 13 - DNA, 22 bp, Artificial Sequence
  Primer
aggagacttatgtcaaatctct

SEQ ID NO: 14 - DNA, 16 bp, Artificial Sequence
  Primer
gtttgggttctgcagt

SEQ ID NO: 15 - DNA, 20 bp, Artificial Sequence
  Primer
gactcctggtagcttgaaca

SEQ ID NO: 16 - DNA, 19 bp, Artificial Sequence
  Primer
acctactcaaagcagcatc

SEQ ID NO: 17 - DNA, 20 bp, Artificial sequence
  Primer
gcttctagtgtggtcttcac

SEQ ID NO: 18 - DNA, 20 bp, Artificial Sequence
  Primer
ggagtagaccgagacatgaa

SEQ ID NO: 19 - DNA, 21 bp, Artificial Sequence
  Primer
attttagttcacccgtaaatc

SEQ ID NO: 20 - DNA, 20 bp, Artificial Sequence
  Primer
cgtgaaacctggaaagaaca

SEQ ID NO: 21 - DNA, 20 bp, Artificial Sequence
  Primer
ccagatccatacagtcgaga

SEQ ID NO: 22 - DNA, 21 bp, Artificial Sequence
  Primer
mccgtcacagcaagctatgaa

SEQ ID NO: 23 - DNA, 19 bp, Artificial Sequence
  Primer
gtcaggcaaaagctccctg

SEQ ID NO: 24 - DNA, 20 bp, Artificial Sequence
  Primer
cctcaagctctttgtacgtt

SEQ ID NO: 25 - DNA, 23 bp, Artificial Sequence
  Primer
accttctcctgctcatcaacaag

SEQ ID NO: 26 - DNA, 20 bp, Artificial Sequence
  Primer
cgcccttttcgattcgcatt

SEQ ID NO: 27 - DNA, 20 bp, Artificial Sequence
  Primer
cccttgaaatcatgcaggta

SEQ ID NO: 28 - DNA, 24 bp, Artificial Sequence
  Primer
mggaattcttgcagatgaaatggt

SEQ ID NO: 29 - DNA, 17 bp, Artificial Sequence
  Primer
cttggagatcgatttga

SEQ ID NO: 30 - DNA, 22 bp, Artificial Sequence
  Primer
tacagctaatgacaccctataa

SEQ ID NO: 31 - DNA, 19 bp, Artificial Sequence
  Primer
acatgtcctcttccaacga

SEQ ID NO: 32 - DNA, 20 bp, Artificial Sequence
  Primer
ccactagtacgtgcatcaga

SEQ ID NO: 33 - DNA, 20 bp, Artificial Sequence
  Primer
atccgagagagcttctctgt

SEQ ID NO: 34 - DNA, 20 bp, Artificial Sequence
  Primer
atccgagagagcttctctgt

SEQ ID NO: 35 - DNA, 21 bp, Artificial Sequence
  Primer
gaagtggtaaccgagagacaa

SEQ ID NO: 36 - DNA, 21 bp, Artificial Sequence
  Primer
aggcgaaacttcatcagagca

SEQ ID NO: 37 - DNA, 19 bp, Artificial Sequence
  Primer
agcaactgttctcgtcttc

SEQ ID NO: 38 - DNA, 20 bp, Artificial Sequence
  Primer
tccgatcacacgagtgttga

SEQ ID NO: 39 - DNA, 20 bp, Artificial Sequence
  Primer
caacacagtacacacaagca

SEQ ID NO: 40 - DNA, 23 bp, Artificial Sequence
  Primer
maccttagtacattgcaatcagt

SEQ ID NO: 41 - DNA, 20 bp, Artificial Sequence
  Primer
tgacatgttaccaagtacca

SEQ ID NO: 42 - DNA, 20 bp, Artificial Sequence
  Primer
caaggtcactgaagacgcaa

SEQ ID NO: 43 - DNA, 23 bp, Artificial Sequence
  Primer
agtaataaccttgcatatgaaag

SEQ ID NO: 44 - DNA, 20 bp, Artificial Sequence
  Primer
acagtgtggtcaccacacat

SEQ ID NO: 45 - DNA, 20 bp, Artificial Sequence
  Primer
gcagatcatggatgatctca

SEQ ID NO: 46 - DNA, 21 bp, Artificial Sequence
  Primer
aggaggcccatatagcaaata

SEQ ID NO: 47 - DNA, 19 bp, Artificial Sequence
  Primer
gactgcgtacgaattcaat

SEQ ID NO: 48 - DNA, 18 bp, Artificial Sequence
  Primer
gctctcctgaaccgcttg

SEQ ID NO: 49 - DNA, 17 bp, Artificial Sequence
  Primer
cacgacgttgtaaaacg

(19) United States
(12) Patent Application Publication
(10) Pub. No.: US 2013/0269065 A1
(43) Pub. Date: Oct. 10, 2013

(54) BLACKLEG RESISTANCE GENE

(71) Applicant: Genyi LI, Winnipeg (CA)

(72) Inventor: Genyi LI, Winnipeg (CA)

(73) Assignee: UNIVERSITY OF MANITOBA, Winnipeg (CA)

(21) Appl. No.: 13/426,010

(22) Filed: Mar. 21, 2012

(51) Int. Cl. C07K 14/415; A01H 5/10; C12N 15/29; A01H 5/00; C12N 15/82; C12N 5/10

(52) U.S. Cl. 800/301; 435/320.1; 435/418; 536/23.6; 530/370

(57) ABSTRACT

Embodiments of the present invention relate to blackleg resistance genes named BLMR1 and BLMR2. Other embodiments of the present invention relate to primers, vectors, DNA, RNA, proteins, cells, seeds, tissues, plants, methods, processes, and uses relating to said gene sequences.

### FIELD OF THE INVENTION

The present invention relates to a gene that encodes for resistance to Leptosphaeria maculans, the cause of blackleg disease in canola. The present invention further pertains to primers, vectors, RNA sequences, and proteins related to said gene. The present invention further relates to cells and plants transformed with said gene and to methods, processes, and uses of said gene.

### BACKGROUND TO THE INVENTION

Blackleg is a serious disease of Brassica spp., such as canola and rapeseed, that can result in significant yield loss in susceptible varieties. The disease is caused by Leptosphaeria maculans, a highly virulent and widespread fungus. Studies of canola fields in Saskatchewan, Canada have found evidence of L. maculans infection in 35-55% of crops surveyed. Average disease incidence values (percentage of plants showing blackleg symptoms) were typically 1% for basal stem cankers and 3% for lesions occurring elsewhere on the stem. The highest incidence values are often observed in crops that had received hail damage. In some fields, L. maculans infects every plant and can reduce seed yield by more than 50%.

Blackleg infections may occur on cotyledons, leaves, stems and pods. The plant is susceptible to blackleg infection from the seedling to pod-set stages. Lesions occurring on the leaves are typically dirty white and are round to irregularly shaped.

On stems, blackleg lesions can be quite variable, but are usually found at the base of the stem, or at points of leaf attachment. Stem lesions may be up to several inches in length, and are usually white or grey with a dark border. Stem lesions may also appear as a general blackening at the base. Severe infection usually results in a dry rot or canker at the base of the stem. The stem becomes girdled and as plants ripen prematurely, the crop is more likely to lodge. Seed may be shriveled and pods shatter easily at harvest, resulting in seed loss.

Blackleg lesions are usually dotted with numerous small, black pycnidia, which are the spore-bearing structures of the fungus. Pycnidia appear as tiny round specks, which may be seen more easily with the aid of a hand lens.

The blackleg fungus can overwinter on infected canola residue and in infected seed. In the spring, the fungus produces fruiting bodies called pseudothecia, on infected canola residue. Ascospores are released from the pseudothecia and become airborne, resulting in long-distance dispersal of the disease to other canola crops. The earlier in the growing season the infection occurs, the greater the likelihood of basal stem canker development and more severe yield loss. Pseudothecia may continue to be produced on infected residue for two more years, or until the infected residue breaks down.

### SUMMARY OF THE INVENTION

The present invention relates to an isolated gene sequence and its homologues that confer resistance in Brassica spp. to blackleg.

According to one embodiment of the present invention, SEQ ID NO: 1 shows the sequence of the blackleg resistance gene BLMR1. SEQ ID NO: 2 and SEQ ID NO: 3 are homologous sequences to SEQ ID NO: 1.

According to one aspect, SEQ ID NO: 4 shows the predicted amino acid sequence of a protein expressed by BLMR1 blackleg resistance gene.

The present invention further relates to primers, vectors, DNA, RNA, proteins, cells, seeds, tissues, plants, methods, processes, and uses relating to said gene sequences.

### BRIEF DESCRIPTION OF THE DRAWINGS

The present invention will be described in conjunction with reference to the following drawings in which:

FIG. 1 shows a comparison of blackleg-susceptible canola cultivar ‘Westar’ and transgenic ‘Westar’ with a blackleg disease resistance gene, after infection with a L. maculans culture.

### DETAILED DESCRIPTION OF THE INVENTION

The present invention relates to a gene sequence isolated from a blackleg-resistant cultivar of canola.

According to one embodiment of the present invention, SEQ ID NO: 1 shows the sequence of a first blackleg resistance gene BLMR1.

According to one aspect, SEQ ID NO: 4 shows the predicted amino acid sequence of a protein expressed by BLMR1.

The present invention relates to sequences having at least 60%, preferably at least 70%, more preferably at least 80%, even more preferably at least 90%, even more preferably still at least 95% homology with one or more of the sequences set forth in SEQ ID NO: 1, SEQ ID NO: 2, and SEQ ID NO: 3.

The present invention relates to RNA transcribed from, or having a complementary sequence to one or more of the sequences set forth in SEQ ID NO: 1, SEQ ID NO: 2, and SEQ ID NO: 3.

The present invention relates to primers comprising one or more of the sequences set forth in SEQ ID NO: 1, SEQ ID NO: 2, and SEQ ID NO: 3.

The present invention relates to expression vectors comprising one or more of the sequences set forth in SEQ ID NO: 1, SEQ ID NO: 2, and SEQ ID NO: 3. Suitable vectors include, but are not limited to binary vectors.

The present invention relates to proteins which provide blackleg resistance. For example, the present invention provides proteins or protein fragments having a high degree of homology with the amino acid sequence set forth in SEQ. ID NO. 4, said proteins providing blackleg resistance.

The present invention relates to methods for transforming cells with one or or more of the sequences set forth in SEQ ID NO: 1, SEQ ID NO: 2, and SEQ ID NO: 3. The cells may be transformed in any suitable manner and techniques well known to those skilled in these arts.

The present invention relates to tissues comprising cells transformed with one or both of the sequences set forth in SEQ ID NO: 1 and SEQ ID NO: 2. Preferably the cells are transformed with SEQ ID NO. 1 only, or alternatively, with SEQ ID NO:1 and SEQ ID NO: 2, or alternatively with SEQ ID NO: 1 and SEQ ID NO: 3, or alternatively with SEQ ID NO: 2 and SEQ ID NO: 3. Preferably the transformed cells are from Brassica sp. such as exemplified by canola, mustard, rapeseed and the like.

The present invention relates to seeds comprising cells transformed with one or more of the sequences set forth in SEQ ID NO: 1, SEQ ID NO: 2, and SEQ ID NO: 3. Preferably the cells are transformed with SEQ ID NO. 1 only, or alternatively, with SEQ ID NO:1 and SEQ ID NO: 2, or alternatively with SEQ ID NO: 1 and SEQ ID NO: 3, or alternatively with SEQ ID NO: 2 and SEQ ID NO: 3. Preferably the seeds are from Brassica sp. such as exemplified by canola, mustard, rapeseed and the like.

The present invention relates to plants comprising cells transformed with one or both of the sequences set forth in SEQ ID NO: 1 and SEQ ID NO: 2. Preferably the cells are transformed with SEQ ID NO. 1 only, or alternatively, with SEQ ID NO:1 and SEQ ID NO: 2, or alternatively with SEQ ID NO: 1 and SEQ ID NO: 3, or alternatively with SEQ ID NO: 2 and SEQ ID NO: 3. Preferably the plants are Brassica sp. cultivars such as exemplified by canola, mustard, rapeseed and the like.

The present invention relates to the use of one or more of the sequences set forth in SEQ ID NO: 1, SEQ ID NO: 2, and SEQ ID NO: 3, or sequences homologous thereto, for providing resistance to blackleg disease.

The present invention relates to the use of the amino acid sequence set forth in SEQ. ID. NO. 4 or active fragments thereof, for providing resistance to blackleg disease.

The present invention relates to the use of the present DNA sequences for developing molecular markers for genes which may encode proteins that provide blackleg resistance.

The present invention relates to the use of the present DNA sequences for identifying genes which may encode proteins which provide blackleg resistance, alternatively for gene pyramiding, and further alternatively for eliminating unwanted flanking regions.

### EXAMPLES

### Example 1

The canola cultivar ‘Surpass 400’ was released as a blackleg resistant cultivar containing one or more blackleg resistance genes. This study focused on the mapping and cloning of a blackleg resistance gene from this cultivar through map-based cloning strategy. A consensus map was developed using SRAP (sequence related amplified polymorphism) markers and a double haploid (DH) population developed from a cross of ‘Westar’ and ‘Zhongyou 821’. F2 and BC2 and BC3 individuals of the ‘Westar’×‘Surpass 400’ cross were used to follow the segregation of disease resistance. One Mendelian gene controlled the disease resistance to blackleg as shown by trait segregation. Starting with an anchoring marker on the ultra-density map, different markers including SNP, SSR and SCAR markers were developed and used to screen over 10,000 BC3 individuals to narrow down the blackleg disease resistance gene in a 15-kb region. One gene candidate found in the 15-kb region was used to do complementary transformation. After introducing the candidate gene into the blackleg disease susceptible canola cultivar ‘Westar’, this cultivar became equally blackleg resistant to the cultivar ‘Surpass 400’.

### Preparation of Constructs, Transformation and Regeneration

A modified binary vector pBI121U was used. The 5′ universal uracil primer “5′-GGAGTTAAU+” was added to the forward primer. The 3′ universal reverse primer tail “5-GGTCTTAAU+” was added to the reverse primer. After PCR with these two primers, the whole gene including promoter and coding regions was joined into pBI121U to have a plant transformation construct. Vector pBI121u was first digested with PacI and then, with the subsequent nicking enzyme, Nt.BbvCI (New England Biolabs Ltd., Pickering, ON, CA). A 10 μl PCR fragments were digested with 1 U USER enzyme (New England Biolabs Ltd., Pickering, ON, CA). The insert mixture, mixed with vector DNA was incubated 20 min at 37° C. followed by 20 min at 25° C. and finally transformed into chemically competent E. coli DH10B cells. The plasmids of positive clones were isolated and sequenced to confirm the accuracy of the sequence. Finally, the construct was electro-transformed into Agrobacterium tumefaciens strain GV3101 and used for B. napus transformation.

Plant transformation followed the protocol described by Moloney et al. (1989, Plant Cell Rep 8: 238-242). B. napus canola cultivar ‘Westar’ is susceptible to blackleg disease and was used to perform complementary transformation. The seeds of ‘Westar’ cultivar were surface-sterilized for 15 min 4% sodium hypochlorite with 0.1% Tween 20 added as a surfactant. Then, the seeds were washed thoroughly with sterile distilled water and germinated on 1/2 MS (Sigma-Aldrich Canada Ltd., Oakville, ON, CA) basal medium with 10 g L⁻¹ sucrose. Hypocotyls were harvested from 4-5 day old seedlings and cut into 4-6 mm long pieces and placed onto MS medium and incubated 3 days at 25° C. Agrobacterium cells were prepared by culturing overnight on shakers at 28° C. in LB medium with appropriate antibiotics, after which the cells were pelleted and re-suspended in the same volume of liquid hormone-free MS with 30 g L⁻¹ sucrose medium. The canola hypocotyl tissue pieces were collected and mixed thoroughly with 10-time dilution of Agrobacterium suspension with hormone-free MS with 30 g L⁻¹ sucrose medium. The excess fluid was discarded and the tissue pieces were co-cultured with Agrobacterium on MS medium for 5 days. The explants were transferred to MS medium with 20 mg L⁻¹ kanamycin for culturing. After a further 2 weeks, the explants were transferred to fresh MS medium. The first shoots developed after 3˜4 weeks. The developing green shoots were transferred to MS medium and the elongated shoots were transferred to 1/2MS medium. The rooted shoots were transferred to a soil-less growing mix and grown in plant growth chamber.

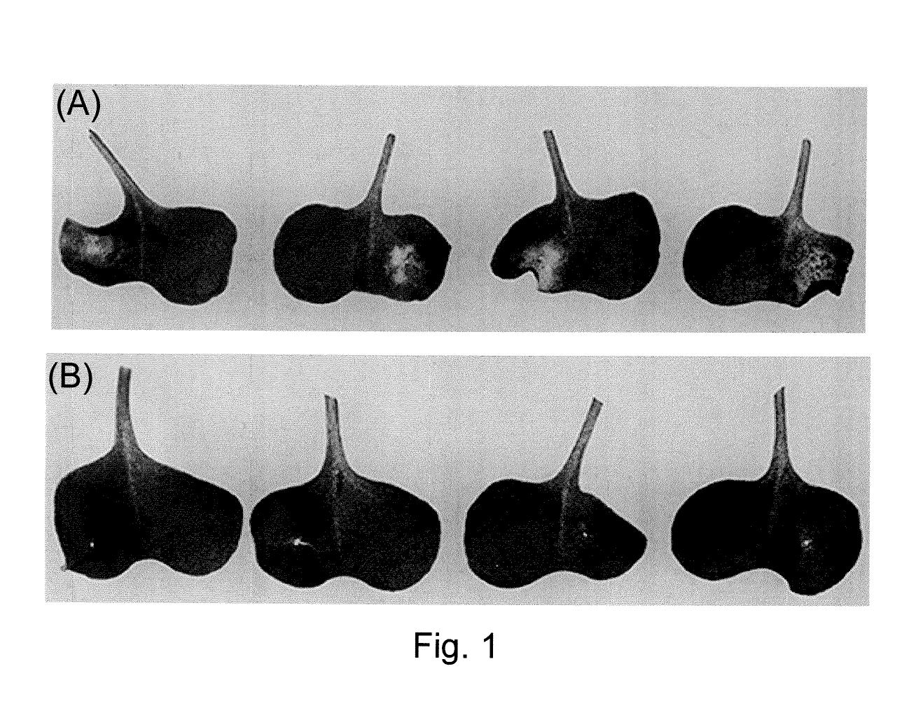

After harvesting, T1 seeds were planted and inoculated with blackleg pathogen. Cotyledons were punctured with sharp pointed forceps. Ten μl of spore suspension was placed on each puncture. The plants were kept at room temperature with light overnight for recovery. The plants were then placed in a controlled growth chamber (14 hrs light at 24° C. during day time and 20° C. at night). In about 12 days, disease symptoms were fully developed, and the disease severity was rated. Disease severity ratings of 0 to 4 were classified as resistant while ratings of 5 to 9 were classified as susceptible. The cultivar ‘Westar’ was used as control for every inoculation run. The testing results showed that the susceptible ‘Westar’ was changed into a blackleg-resistant resistant transgenic ‘Westar’ (FIG. 1).

### Example 2

### Mapping Populations and Blackleg Isolates

A resistant B. napus canola cultivar ‘Surpass 400’ and a susceptible B. napus canola cultivar ‘Westar’ were used to produce mapping populations. A total of 908 F₂ and 2,992 F₃ individuals were inoculated and screened with a blackleg isolate 87-41 at the cotyledon stage. Two F₃ lines showing different interactions with the blackleg isolate 87-41 were backcrossed to ‘Westar’. Sixteen F₃BC₁ lines were produced to observe phenotypic segregation. After two genes on linkage group N10 were separated, fine mapping was performed with 1513 F₃BC₂ individuals that segregate at the locus corresponding to a strong resistance phenotype and with 800 F₃BC₂ individuals that segregate at the second locus conferring a weak resistance phenotype respectively.

### Preparation of Blackleg Isolate Suspension

Pycnidial inoculum of the blackleg isolate 87-41 was prepared with a method modified from the teaching of Mengistu et al. (1991, Plant Dis. 75:1279-1282). The modifications were as follows: The cotyledons with lesions were collected and washed three times in sterilized distilled water in a laminar hood. The cotyledons were then treated with 15% (V/V) bleach for 20 minutes with occasional agitation. After three 2-minute washes with sterilized water, the cotyledons were transferred to Petri dishes with V8 agar medium (250 ml V8 juice, 0.5 g CaCO₃ and 15 g granulated agar per litre). The dishes were placed in a temperature and light controlled growth chamber. After incubating for a week, the cotyledons were full of black pycnidia and sometimes pink pycnidiospores were released. The spores were discharged by washing and scraping the agar surface with sterilized glass. The blackleg inoculum concentration was adjusted with distilled water to 2×107 spores/ml from the stock solution.

### Phenotype Determination by Inoculation

Cotyledons of individual plants were punctured with sharp pointed forceps. Ten μl of spore suspension was placed on each puncture. The plants were kept at room temperature overnight for recovery. The plants were then placed in a controlled growth chamber (14 hrs light at 20° C. during day time and 18° C. at night). In about 12 days, disease symptoms were fully developed, and the disease severity was rated according to the classification of 0-9 taught by Chen and Fernando (2005, Eur. J. Plant Pathol. 114: 41-52). Disease severity ratings of 0 to 4 were classified as resistant while ratings of 5 to 9 were classified as susceptible. The cultivars ‘Westar’ and ‘Surpass 400’ and their F1 progeny were used as controls for every inoculation run.

### DNA Extraction and SRAP Marker Development

A modified CTAB extraction procedure as taught by Li and Quiros (2001, Theor. Appl. Genet. 103: 455-461) was used to extract DNA. SRAP was performed as described by Sun et al. (2007, Theor. Appl. Genet. 114: 1305-1317). A five fluorescent dye color set, ‘6-FAM’, ‘VIC’, NED', ‘PET’ and ‘LIZ’, were used for signal detection using an ABI 3100 Genetic Analyzer (ABI, Toronto). The ‘LIZ’ color was used for the size standard, while the other four colors were used to label SRAP primers. The ultradense genetic recombination map with 13551 SRAP markers that was constructed with 58 DH lines from a cross of ‘Westar’ and ‘Zhongyou 821’ was used to develop SRAP markers that were linked to the resistance gene. To use this map, the mapping population of ‘Westar’ and ‘Surpass 400’ was screened with the same primer sets as used for the ultradense map construction. DNA samples from 8 resistant plants and 8 susceptible plants were used to perform an initial round of SRAP marker analysis. After a molecular marker was found to co-segregate with disease resistance, DNA samples from 64 resistant plants and 64 susceptible plants were tested to confirm the linked SRAP markers that were used to find the corresponding SRAP molecular marker on the ultradense map. After anchoring the molecular markers linked to the resistance gene on the ultradense SRAP map, the SRAP molecular markers flanking the anchoring marker were used to find increasingly closer SRAP markers.

### SRAP Marker Sequencing and Finding Arabidopsis Synteny

SRAP PCR products were separated with sequencing gels. The gels were stained with a silver staining kit (Promega Corp, Madison, Wis., USA). The target markers were identified by comparing the band patterns with the marker patterns that were produced with the ABI 3100 Genetic Analyzer (Applied Biosystems, Carlsbad, Calif., USA). DNA was eluted as described in by Sambrook and Maniatis (2001, Molecular Cloning, A Laboratory Manual, Cold Spring Harbour Laboratory Press, Cold Spring harbour, NY, USA). The DNA was reamplified and compared with the original SRAP profile to confirm the right position by running the PCR products on an ABI 3100 Genetic Analyzer. The confirmed DNA products were sequenced with a BigDye Terminator v3.1 kit (Applied BioSystems).

BLAST analysis of the marker sequences was performed with the TAIR Arabidopsis database (http://www.arabidopsis.org). Sequences of some SRAP markers were found to be homologous to Arabidopsis genes.

Development of genome specific SCAR and SNP

BAC clone sequences on linkage group R10 of a B. rapa genetic map (http://www.brassica-rapa.org/BRGP/geneticMap.jsp) that corresponded to N10 in B. napus were selected to develop genome specific codominant molecular markers. In total, nine BAC clones were selected and primers were designed according to the BAC sequences. First, these primers were used to amplify B. oleracea DNA to obtain corresponding sequences in the C genome. Second, new primers that are located at the sequence difference positions between B. rapa and B. oleracea were used to find the primer combinations that amplify only the A genome DNA in B. napus. Then, the A genome specific primers were used to amplify ‘Surpass 400’ and ‘Westar’ to identify sequence insertion/deletion and single nucleotide polymorphism. Finally, those sequence differences were developed into sequence characterized amplified polymorphism (SCAR) or single nucleotide polymorphism (SNP) markers.

### SCAR and SNP Detection

For SCAR marker detection, a M13 primer sequence (CACGACGTTGTAAAACGAC) was added to one of two genome specific primers of SCAR markers. The M13 primer was labeled with four of five color fluorescent dyes, 6-FAM, VIC, NED, PET and LIZ (internal standard) (Applied BioSystems). The PCR reactions for SCAR marker detection were set up in 10 μl mixture containing two genome specific primers and one labeled M13 primer were included in the PCR cocktail. The concentrations for one genome specific primer without the M13 tail and the labeled M13 were 0.15 μM and the concentration of the genome specific primer with M13 tail was 0.05 μM. Other components in the reaction mixture included 50 ng genomic DNA, 1×PCR buffer, 0.375 mM dNTP, 1.5 mM MgCl and 1 unit Taq. A touch-down PCR running program (94° C. 3 min; 94° C., 1 min, 57° C. with −0.8° C. each cycle, 1 min and 72° C. 1 min for 6 cycles; 94° C., 1 min, 57° C., 1 min and 72° C., 1 min for 30 cycles) was used to run SCAR marker reactions. The PCR products were separated in ABI 3100 Genetic analyzer. The data were collected and analyzed with ABI GenScan software and further transferred into images for scoring using Genographer software available at http://hordeum.oscs.montana.edu/genographer.

SNP detection followed the procedure taught by Rahman et al. (2008, in A. H. Paterson (Ed.) Genetics and Genomics of Cotton, Springer, 3:1-39). The genome specific primers were used to obtain PCR products containing SNP positions. PCR reactions were performed in a 10 μl mixture containing 50 ng of genomic DNA, 375 μM dNTP, 0.15 μM of each primer, 1×PCR buffer, 1.5 mM MgCl₂ and 1 Unit of Taq polymerase. The PCR program was 94° C. for 3 mM, followed by 35 cycles of 94° C. for 1.0 mM, 55° C. for 1.0 min, 72° C. for 1.0 min and final extension 72° C. for 10 min. In SNP detection, detection primers were added with a poly A tail to obtain different sizes of products that were used to pool samples before separation. The SNaPshot multiplex kit (ABI, California) was used following the instruction in the kit. The SNaPshot products were pooled first and 2 μl pooled DNA was mixed with 8 μl formamide containing 120 LIZ size standards (Applied BioSystems). Then, the DNA fragments were analyzed with an ABI 3100 Genetic Analyzer. Genotypes were scored manually, using peak color verification.

All primers used in this study were list in Table 1.

TABLE 1
Primers for genome-specific co-dominant molecular SCAR, SNP and SRAP
markers

| Marker | Marker | Primer | | |
|---|---|---|---|---|
| name | type | name | SEQ ID NO: | Primer sequence 5′-3′ |
| 80A08a | SNP | 80A08A | SEQ ID NO: 5 | GGTATCGCATTCTGTGACTA |
| | | 80A08B | SEQ ID NO: 6 | GGAGATGTGCTTCAACGTGA |
| | | 80A08R | SEQ ID NO: 7 | A₂₈ACATTCTTGGGCCGTAGG |
| 80E24a | SNP | 80E24A | SEQ ID NO: 8 | GACAAACACAATGGACTCAA |
| | | 80E24B | SEQ ID NO: 9 | GAGGTAGAGAAAGACGAAGA |
| | | 80E24R | SEQ ID NO: 10 | A₂₀ATCGTTTAAGGAATGTGCCAA |
| 9B23a | SNP | 9B23A1 | SEQ ID NO: 11 | CCACAGTTTCTGGAGAC |
| | | 9B23B1 | SEQ ID NO: 12 | GTAGCAAAGGAATCAATTAA |
| | | 9B23R1 | SEQ ID NO: 13 | A₁₉AGGAGACTTATGTCAAATCTCT |
| 9B23b | SNP | 9B23A2 | SEQ ID NO: 14 | GTTTGGGTTCTGCAGT |
| | | 9B23B2 | SEQ ID NO: 15 | GACTCCTGGTAGCTTGAACA |
| | | 9B23R2 | SEQ ID NO: 16 | A₂₂ACCTACTCAAAGCAGCATC |
| 9B23c | SNP | 9B23A3 | SEQ ID NO: 17 | GCTTCTAGTGTGGTCTTCAC |
| | | 9B23B3 | SEQ ID NO: 18 | GGAGTAGACCGAGACATGAA |
| | | 9B23R3 | SEQ ID NO: 19 | A₂₀ATTTTAGTTCACCCGTAAATC |
| 87B10a | In/del | 87B10A | SEQ ID NO: 20 | CGTGAAACCTGGAAAGAACA |
| | | 87B10B | SEQ ID NO: 21 | CCAGATCCATACAGTCGAGA |
| | | 87B10R | SEQ ID NO: 22 | M₁₃MCCGTCACAGCAAGCTATGAA |
| 5200 | SNP | 5200A | SEQ ID NO: 23 | GTCAGGCAAAAGCTCCCTG |
| | | 5200B | SEQ ID NO: 24 | CCTCAAGCTCTTTGTACGTT |
| | | 5200R | SEQ ID NO: 25 | A₁₃ACCTTCTCCTGCTCATCAACAAG |
| 6200 | In/del | 6200A | SEQ ID NO: 26 | CGCCCTMCGATTCGCATT |
| | | 6200B | SEQ ID NO: 27 | CCCTTGAAATCATGCAGGTA |
| | | 6200R | SEQ ID NO: 28 | M₁₃MGGAATTCTTGCAGATGAAATGGT |
| 1J13a | SNP | 1J13A1 | SEQ ID NO: 29 | CTTGGAGATCGATTTGA |
| | | 1J13B1 | SEQ ID NO: 30 | TACAGCTAATGACACCCTATAA |
| | | 1J13R1 | SEQ ID NO: 31 | A₂₀ACATGTCCTCTTCCAACGA |
| 1J13b | SNP | 1J13A2 | SEQ ID NO: 32 | CCACTAGTACGTGCATCAGA |
| | | 1J13B2 | SEQ ID NO: 33 | ATCCGAGAGAGCTTCTCTGT |
| | | 1J13R2 | SEQ ID NO: 34 | A₂₀ATTGTTATACTCAGACCCACC |
| 10B23a | SNP | 10B23A | SEQ ID NO: 35 | GAAGTGGTAACCGAGAGACAA |
| | | 10B23B | SEQ ID NO: 36 | AGGCGAAACTTCATCAGAGCA |
| | | 10B23R | SEQ ID NO: 37 | A₁₁M₁₃AGCAACTGTTCTCGTCTTC |
| 12D09a | In/del | 12D09A | SEQ ID NO: 38 | TCCGATCACACGAGTGTTGA |
| | | 12D09B | SEQ ID NO: 39 | CAACACAGTACACACAAGCA |
| | | 12D09R | SEQ ID NO: 40 | M13MACCTTAGTACATTGCAATCAGT |
| 43M07a | SNP | 43M07A | SEQ ID NO: 41 | TGACATGTTACCAAGTACCA |
| | | 43M07B | SEQ ID NO: 42 | CAAGGTCACTGAAGACGCAA |
| | | 43M07R | SEQ ID NO: 43 | A₁₈AGTAATAACCTTGCATATGAAAG |
| 81M20a | SNP | 81M20A | SEQ ID NO: 44 | ACAGTGTGGTCACCACACAT |
| | | 81M20B | SEQ ID NO: 45 | GCAGATCATGGATGATCTCA |
| | | 81M20R | SEQ ID NO: 46 | A₁₀AGGAGGCCCATATAGCAAATA |
| R278 | SRAP | EM1 | SEQ ID NO: 47 | GACTGCGTACGAATTCAAT |
| | | BG28 | SEQ ID NO: 48 | GCTCTCCTGAACCGCTTG |

M₁₃, 5′-CACGACGTTGTAAAACG-3′ (SE ID NO: 49)
Aₙ: n means the number of nucleotide ‘A’.

### Segregation of the Blackleg Resistance in the Mapping Populations

In the mapping populations of ‘Westar’ and ‘Surpass 400’, 908 F₂ individuals and 12 plants from each derived F₃ line were inoculated with a blackleg isolate 87-41. Among the F₂ population, there were 693 resistant plants and 215 susceptible plants showing a 3:1 segregation ratio (X2 test, p-value=0.36). In the F₃ population, all plants in each of 232 F₃ lines were resistant, all plants in each of 209 F₃ lines were susceptible, and the rest of the F₃ lines showed segregation in these twelve tested plants in each line, showing a 1:2:1 segregation ratio (X2 test, p-value=0.38). The segregation of the resistance gene in the F₂ and F₃ generations of the ‘Westar’ and ‘Surpass 400’ cross showed a 3:1 segregation ratio in the F₂ and a 1:2:1 segregation ratio in the F₃ families, suggesting that one dominant resistance gene controls the blackleg resistance in ‘Surpass 400’.

### Identification of Linked SRAP Markers on a Consensus Map

For marker analysis and gene mapping, DNA samples were prepared from all 908 F₂ plants, from one plant from each of these 232 resistant F₃ lines, from two plants from each of these 209 susceptible F₃ lines, and from two to four susceptible plants from each segregated F₃ lines; in total 2992 F₃ plants. For primer screening, 8 susceptible plants and 8 resistant plants from the F2 mapping population were used to run SRAP molecular markers. Three hundred and eighty four primer pairs were used for the initial screening and two SRAP markers R269 and G278 were found to co-segregate with the resistance gene in 16 plants tested. By comparing these two SRAP markers with the SRAP molecular markers on the ultradense genetic recombination map, it was found that R269 corresponded to SRAP marker 1217Ar 269 on N10 linkage group (for information on SRAP marker 1217Ar 269, refer to Sun et al. 2007, Theor. Appl. Genet. 114: 1305-1317), but there was no corresponding SRAP marker to G278. After searching the polymorphism of the 32 SRAP markers flanking 1217Ar269 marker on N10 linkage group with the ‘Westar’ and ‘Surpass 400’ segregation populations, 210Ay442 and 1128BG275 on the map were found to co-segregate with the blackleg resistance gene.

### Sequencing of SRAP Markers and Identification of Synteny in Arabidopsis Genome

The linked SRAP molecular markers G278, 1217Ar269, 210Ay442 and 1128BG275 were sequenced. After BLAST analysis against the Arabidopsis database (http://www.arabidopsis.org), the sequence of SRAP marker 1128BG275 was found to have a match to At5g18840 (201 nt, E-value: 2e-09) and that of G278 a match to At5g57345 (192 nt, E-value: 3e-29) in Arabidopsis, respectively. Unfortunately, there were no solid hits in Arabidopsis for the sequences of the remaining two SRAP markers 1217Ar269 and 210Ay442. The corresponding genes to the SRAP marker sequences were located in two syntenic regions in Arabidopsis and according to the comparative genetic information, a corresponding region on linkage group R10 of B. rapa was found (http://www.brassica-rapa.org/BRGP/geneticMap.jsp).

1. An isolated nucleic acid molecule comprising a nucleotide sequence set forth in one of SEQ ID NO: 1 or comprising a nucleotide sequence that exhibits from about 80% to 100% identity with SEQ ID NO: 1, and

2. An isolated nucleic acid molecule comprising a nucleotide sequence set forth in one of SEQ ID NO: 2 or comprising a nucleotide sequence that exhibits from about 80% to 100% identity with SEQ ID NO: 2.

3. An isolated nucleic acid molecule comprising a nucleotide sequence set forth in one of SEQ ID NO: 3 or comprising a nucleotide sequence that exhibits from about 80% to 100% identity with SEQ ID NO: 3.

4. An isolated protein molecule comprising an amino acid sequence set forth in SEQ ID NO: 4, said protein molecule effective for inhibiting Leptosphaeria maculans.

5. An expression vector comprising one of the nucleic acid molecule of claim 1, the nucleic acid molecule of claim 2, the nucleic acid of claim 3, and combinations thereof.

6. A plant cell transformed with the expression vector of claim 5.

7. The plant cell according to claim 6, wherein the cell was isolated from a Brassica sp. plant cultivar.

8. A seed comprising the plant cell according to claim 7.

9. A plant comprising the plant cell according to claim 7.

10. A plant cell, seed, or plant comprising the protein molecule according to claim 4.

11. A composition comprising the protein molecule according to claim 4.

12. Use of the protein molecule according to claim 4 for providing a plant with resistance to blackleg disease.

13. Use of the composition of claim 10 for providing a plant with resistance to blackleg disease.
